(19) United States
(12) Patent Application Publication
(10) Pub. No.: US 2021/0386537 A1
(43) Pub. Date: Dec. 16, 2021

(54) Intraocular Lens Assembly

(71) Applicant: Abhijeet Khake, PUNE (IN)

(72) Inventor: Abhijeet Khake, PUNE (IN)

(21) Appl. No.: 17/292,175

(22) PCT Filed: Nov. 12, 2019
(86) PCT No.: PCT/IN2019/050835

§ 371 Date: May 7, 2021

(30) Foreign Application Priority Data
Nov. 12, 2018 (IN) 201821042478

(51) Int. Cl. A61F 2/16

(52) U.S. Cl. CPC A61F 2/16; A61F 2002/1683; A61F 2002/16902; A61F 2002/169

(57) ABSTRACT

An intraocular lens assembly is used in cataract surgery. An intraocular lens assembly (100) has a haptic (102) having a ring (106), a plurality of arcuate arms (108), and a plurality of haptic arm bases (110). Each of the arms (108) is connected with the ring (106) by a respective base (110). An optic (104) has at least two pairs of opposed holes (112). The holes (112) are positioned in the close proximity with an outer peripheral edge of the optic (104). The optic (104) is removably positionable in the haptic (102) by a snap fit lock forming the intraocular lens assembly. The intraocular lens assembly (100) has a first unlocked position wherein haptic (102) is dissembled from the optic (104), and a second locked position wherein the optic (104) is snap fitted in the haptic (102).

### FIELD OF THE INVENTION

The present invention relates to an intraocular lens (IOL) assembly, particularly to a haptic and optic locking arrangement with four point fixation arrangement.

### BACKGROUND OF THE INVENTION

Cataract surgery is the most commonly performed surgery all over the world. In cataract surgery the natural lens which has become opaque is removed and an artificial lens is inserted. The power estimation of the intraocular lens has some limitations, due to which at times there may be refractive surprises after surgery. Refractive surprises can also occur in post laser refractive surgery, high myope and high hyperopic patients undergoing cataract surgery and patients with conical opacity undergoing cataract surgery. Secondly there can be alteration in the refraction of post cataract surgery patients undergoing retinal surgical procedures. Visual quality may also deteriorate if the optic of the lens gets damaged as in glistening or during YAG (yttrium-aluminum-garnet) capsulotomy. To rectify such problems patient will require intraocular lens exchange that has very complicated and difficult procedure and also that can lead to significant tissue manipulations and damage.

Some designs have been known in the prior art in which the lens power can be changed. These prior art inventions describe optic which can be separated from the haptic and can be changed. But these inventions either lack a stabilizing or fixing system between the haptic and optic system i.e. locking system, and if a locking system is present it is done by separating the optic in 2 to 3 parts. U.S. Pat. No. 8,066,768 to Theodore Werblin has proposed a multi-component intraocular lens system where the base lens is attached with haptic and the top and mid lenses are assembled on top of it. In this cited patent, the top and mid lenses include projections designed for locking them into place with flanges of the base lens. This elaborated IOL has the risk of cellular deposits and interlenticular membrane formation at the level of the interfaces causing visual impairment over time.

These multi-component intraocular lens systems, due to their complicated design needs a lot of maneuvering and tissue handling while insertion and exchange. Further as optic of these designs are in 2 or 3 parts, there can be deterioration in image quality if alignment is not perfect or if the lens alignment changes even slightly after insertion in future. Furthermore, in these designs integrating all the components of optic and locking inside the eye is technically difficult, also aligning the optic lenses to get perfect outcome is not easy.

U.S. Pat. No. 6,027,531 to Tassignon has disclosed intraocular lens and method for avoiding secondary posterior capsular opacification. In this patent, anterior and posterior capsulorhexis are made and the rhexis margins are trapped in the groove between the outer anterior and posterior flange of the haptic plate. This type of insertion of IOL is called ‘bag in the lens’, as the bag is trapped inside the lens, instead of the conventional ‘lens in the bag’ wherein the IOL is placed inside the capsular bag. Further in this bag-in-the-lens technique the optic is pushed between the inner anterior and posterior flange. Here the disadvantages of the IOL system are that it requires anterior as well as posterior capsulorhexis and the anterior and posterior capsular rims are hooked in the notch between outer anterior and posterior flanges of the haptic. This will cause tremulous movement of the haptic plate on eye movement and vision disturbance. Secondly it is technically difficult to adjust and fix the haptic in both the anterior and the posterior capsules. It may cause damage to capsule and may cause vitreous disturbance and vitreous loss. Also, the shape of anterior and posterior capsulorhexis should be exactly circular. Furthermore the rotational stability of the optic inside the inner flanges of the ring is less, as it is a curved surface and removal of the optic is difficult as there is no system to facilitate removal. Also, as there is no posterior capsule, optic can slip down into the vitreous while inserting requiring a vitreous surgery.

Thus, there is a need for an intraocular lens (IOL) assembly, wherein optic can be changed whenever required with ease and minimal tissue manipulation and wherein there is a simple and secure locking system between the haptic and optic for 360 degree at the haptic optic junction. The locking system should provide anteroposterior and rotational stability along with slip-free and split-free removal of the changeable optic. Furthermore, the assembly should have a one-piece optic and should avoid posterior capsulorhexis.

### SUMMARY OF THE INVENTION

An intraocular lens assembly has an optic and a haptic. The haptic has a haptic ring, a plurality of arcuate arms and a plurality of haptic arm bases. Each of the arms is connected with the ring by a respective base. The optic has at least two pairs of opposed holes or notches. The holes or notches are positioned in the close proximity with an outer peripheral edge of the optic. The optic is removably positionable in the haptic through a snap fit lock forming the intraocular lens assembly. The intraocular lens assembly has each of the arcuate arms that have a first end that includes a rounded tip, and the respective second end that is connected to the base.

The haptic includes a first groove, the first groove is configured to run along the inner circumference of the haptic. The first groove includes a wedge and a step. The wedge includes a posterior wall and an anterior wall which meet at apex on inner circumference of haptic. The step has an anterior vertical first wall, a horizontal second wall and a posterior vertical wall.

The optic includes a second groove that has a step, a hatch with a planer base, a peripheral wall and a top wall. The step 600 is defined by a peripheral wall 606, the posterior and inner vertical third wall 610, and a horizontal second wall 604. The haptic of assembly has anterior first wall and the horizontal second wall that are perpendicular to each other. The wedge includes a posterior wall and a horizontal wall. The horizontal second wall of the step and posterior third wall of the step are perpendicular to each other.

The second groove 404 is slideably positionable inside the first groove such that the base of the hatch rests on second horizontal wall of haptic. The peripheral anterior wall of the optic apposes to the anterior-outer first wall of the haptic. The vertical wall of optic apposes to posterior and inner vertical third wall of haptic. The top of the hatch touches the posterior wall of the wedge. The optic has notches being positioned in the close proximity with an outer peripheral edge of the optic. The haptic has rounded protrusion along both edges of the arms that provide better fixation and anteroposterior stability of the haptic.

### BRIEF DESCRIPTION OF THE DRAWINGS:

FIG. 1 is a perspective view of the IOL assembly 100 shown in an assembled position in accordance with the present invention;

FIG. 2 is a top view of the IOL assembly 100 of FIG. 1;

FIG. 3 is an exploded view of the haptic and optic of the IOL assembly 100 of FIG. 1;

FIG. 4 is a cross sectional view of the intra ocular lens assembly 100 showing a lock mechanism in accordance with the present invention;

FIG. 5 is a partially enlarged cross sectional view of the haptic of the IOL assembly 100 of FIG. 1;

FIG. 6 is a partially enlarged cross sectional view of the optic of the IOL assembly 100 of FIG. 1;

FIG. 7 is a frontal cross-sectional view of the IOL assembly 100 of FIG. 1;

FIG. 8 is a top perspective of another embodiment of the IOL assembly 100 in accordance with the present invention 100;

FIG. 9 is a top perspective of the optic of the IOL assembly 100 of FIG. 8;

FIG. 9B is a top view of an embodiment of the IOL assembly 100 in accordance with the present invention.

FIG. 10A illustrates operational steps of first embodiment of IOL assembly 100 of FIG. 1;

FIG. 10B illustrates continued operational steps of first embodiment of IOL assembly 100 of FIG. 10A;

FIG. 11A illustrates operational steps of second embodiment the IOL assembly 100 of FIG. 1 to be used when capsular support is weak; and

FIG. 11B illustrates continued operational steps of second embodiment of the IOL assembly 100 of FIG. 11A.

### DESCRIPTION OF THE INVENTION

The foregoing objects of the present invention are accomplished and the problems and shortcomings associated with the prior art, techniques and approaches are overcome by the present invention as described below in the preferred embodiments.

All materials used herein were commercially purchased or prepared from commercially purchased materials as described herein.

Although specific terms are used in the following description for sake of clarity, these terms are intended to refer only to particular structure of the invention selected for illustration in the drawings and are not intended to define or limit the scope of the invention.

References in the specification to “preferred embodiment” means that a particular feature, structure, characteristic, or function described in detail thereby omitting known constructions and functions for clear description of the present invention.

In a preferred embodiment, the present invention relates to an intraocular lens (IOL) assembly having a haptic subassembly and an optic subassembly along with a locking arrangement adapted between them. The IOL assembly has a 360 degree haptic and optic locking arrangement wherein the optic is secured and changeable and/or separably mounted on equidistant four point fixating haptic platform, to provide enhanced anteroposterior and rotational stability for replacement or separation of the optics from the haptic without slipping or splitting thereof.

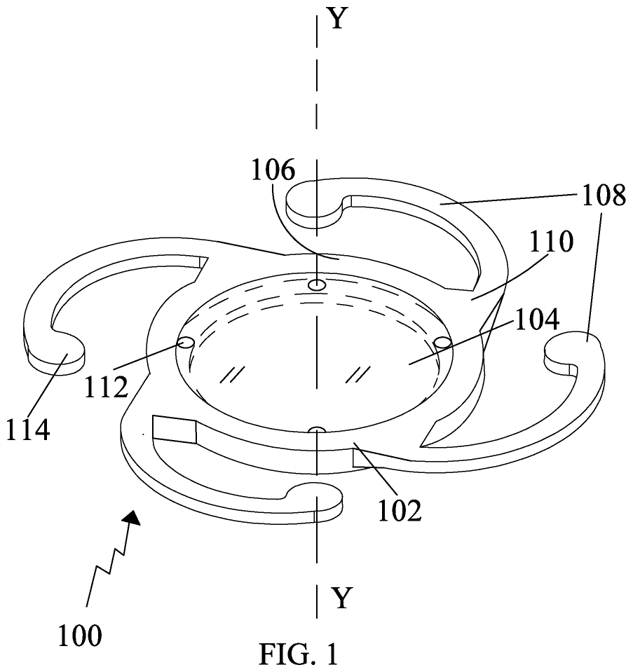

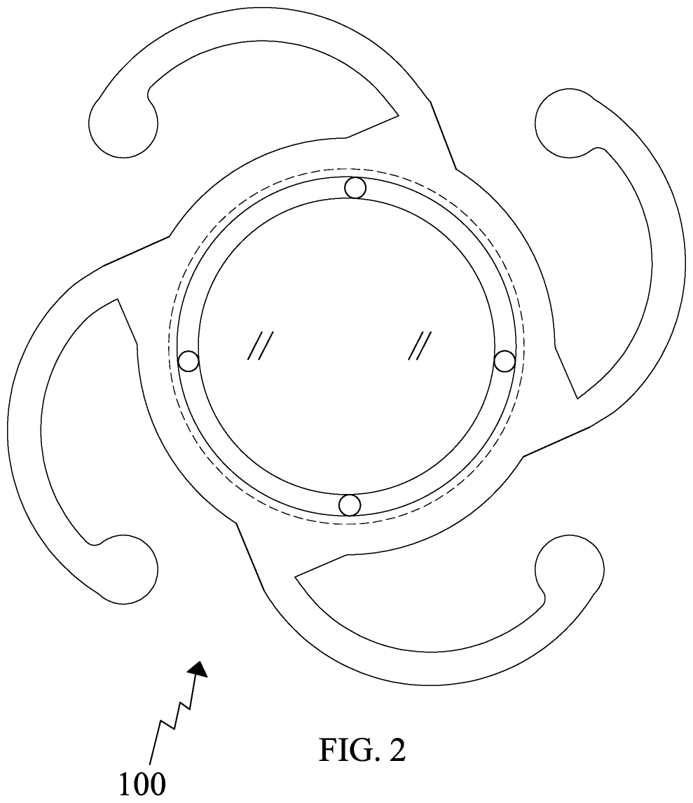

Referring to FIGS. 1 and 2, an intraocular lens assembly 100 in accordance with a preferred embodiment of the present invention is described. The intraocular lens assembly 100 includes a haptic 102, and an optic 104. The optic 104 is removably replaceable in the haptic 102. In this one preferred embodiment the haptic 102 includes a haptic ring 106 and arcuate arms 108. In this preferred embodiment the shape of arms 108 is like the alphabet ‘C’ or like a semicircular loop. Each of the arms 108 is connected with the ring 106 at predefined position by a respective base 110 having predefined shape. The arms 108 are uniformly positioned on the ring 106. The arms 108 has two ends the first end has rounded tip 114 and the second end includes base 110. The arms 108 are permanently connected with the ring 106 through the base 110. In this preferred embodiment of the present invention the bases 110 are preferably triangular in shape. However, the shape of bases 110 may vary in other embodiments of the present invention.

The body of optic 104 is circular and the top and bottom is defined by curved surface. The optic 104 includes pairs of opposed holes 112. In this one embodiment the optic 104 includes two pairs of optic holes 112. These holes 112 are preferably positioned in the close proximity with the outer peripheral edge of the optic 104.

In accordance with the preferred embodiment of the present invention, the material for making haptic 102 and optic 104 is inert, biocompatible, flexible, and foldable material preferably silicon or acrylic. The haptic 102 is opaque or translucent and the optic 104 is transparent.

For example in one embodiment of the present invention the inner diameter of haptic 102 is approximately 5.5 mm and outer diameter is approximately 7.5 mm.

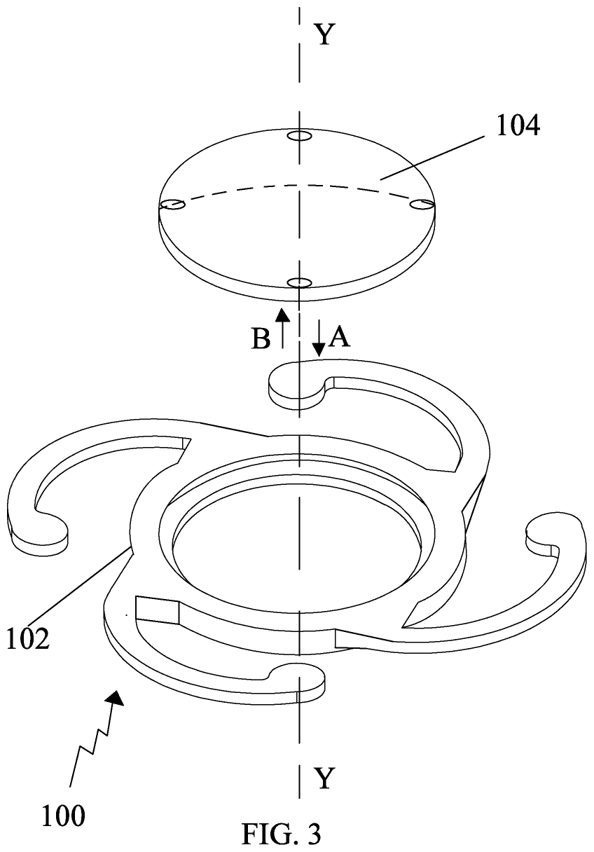

Now referring to FIG. 3, a top perspective view of the haptic optic assembly 100 in a dissembled position is shown. The optic 104 is insertable in the haptic 102 along the axis-Y by moving it in a direction indicated by arrow ‘A’. The optic 104 is removable from the haptic 102 by moving it in the direction of arrow indicated by ‘B’.

The optic 104 is locked in the haptic 102 by a lock. The lock is defined on the inner portion of the haptic ring 106.

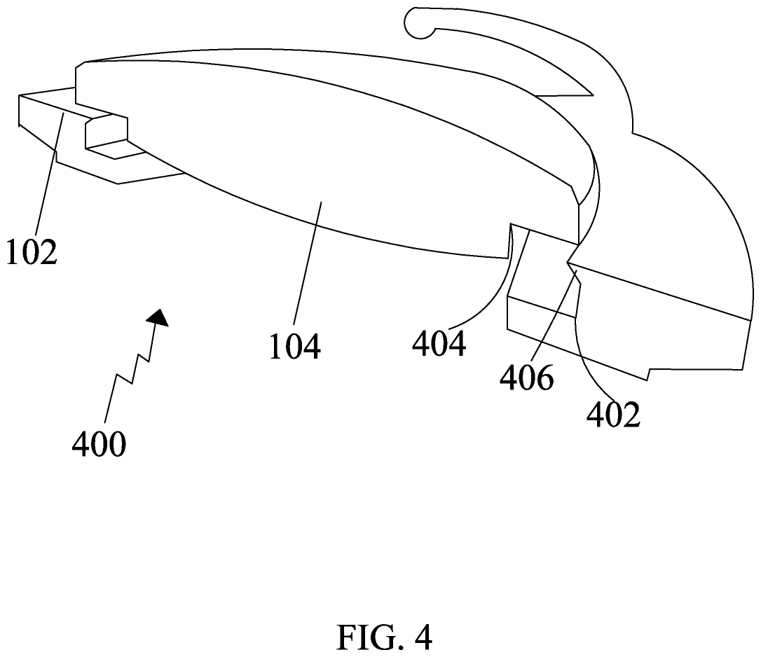

Now referring to FIG. 4 the lock 400 is described. The anterior portion of the haptic 102 has a first groove 402 that runs along the inner circumference of the haptic 102. The posterior portion of the optic 104 has a second groove 404 that runs along the circumference of the optic 104.

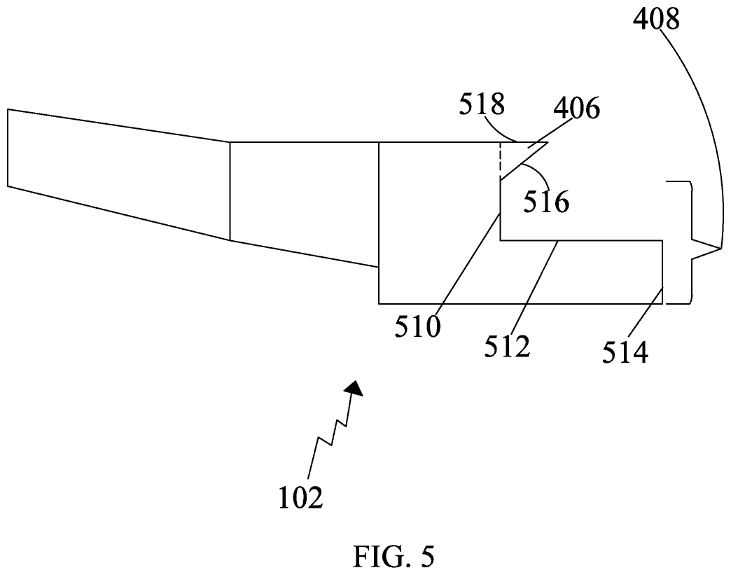

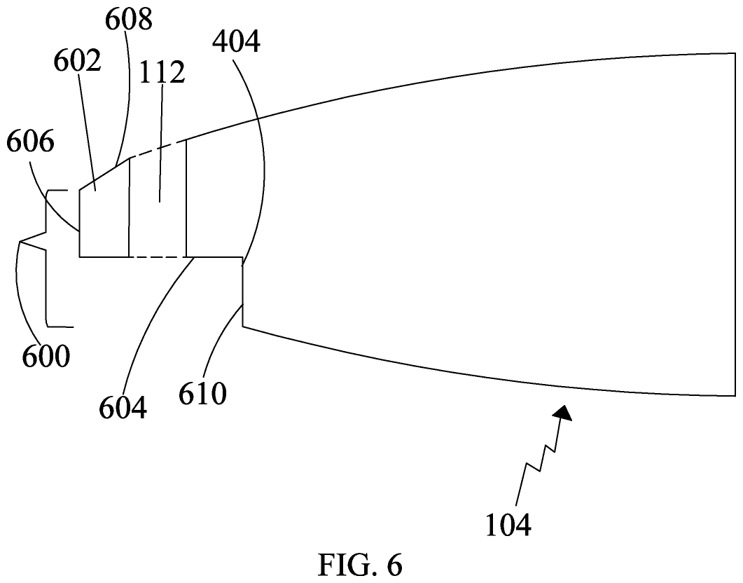

Referring to FIGS. 5 and 6, the first groove 402 of the haptic 102 has a wedge 406 and a step 408. The step 408 is defined by first wall 510, the third wall 514 and a horizontal second wall 512. The second wall 512 joins the two vertical walls 510 and 514. The first wall 510 is preferably anterior-outer vertical wall. The first wall 510 and second wall 512 are approximately perpendicular to each other. Similarly, the second wall 512 and third wall 514 are perpendicular to each other. The third wall 514 is preferably posterior inner and vertical wall.

The wedge 406 includes posterior wall 516 and horizontal anterior wall 518 that guides entry of optic 104. In the preferred embodiment the wedge 406 is triangular in shape and has an inclined posterior wall 516 and horizontal anterior wall 518. The first end of the posterior wall 516 is connected with the first wall 510 of step 408. Similarly, the free end is connected with anterior wall 518. In this preferred embodiment, the positioning of the posterior wall 516 and anterior wall 518 form a wedge 406 of the haptic 102 in accordance with the present invention. It is understood that, the shape and size of the wedge 406 may vary in other embodiments of the present invention.

The second groove 404 includes step 600 that is defined by hatch 602 that has a planer base 604, peripheral anterior wall 606 and top wall 608. The step is defined by peripheral wall 606, the posterior and inner vertical third wall 610 and a horizontal second wall or base 604 joining the two vertical walls.

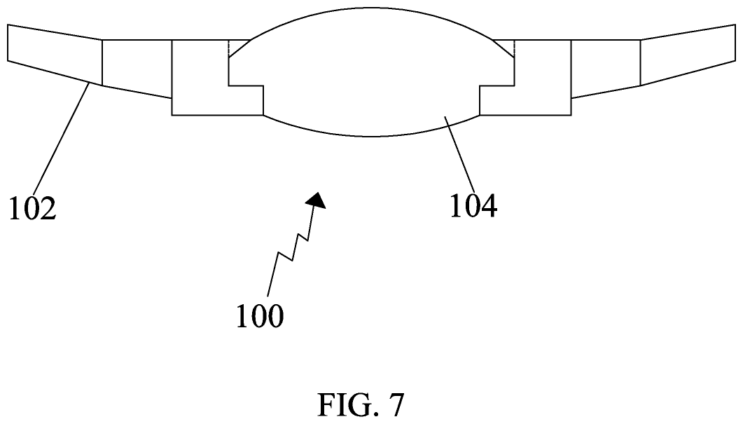

Now referring to FIGS. 4, 5, 6 and 7 the lock 400 in this preferred embodiment is described. The optic 104 is positioned from the top on the haptic 102. By applying a predefined amount of force on the optic 104, the second groove 404 slides inside the first groove 402 such that the base 604 of optic 104 rests on second horizontal wall 512 of haptic 102, and peripheral wall 606 of optic 104 apposes to first wall 510 of haptic 102.The wall 610 of optic 104 apposes to third wall 514 of haptic 102. The top 608 of the hatch 602 touches the posterior wall 516 of the wedge 406.

In this one embodiment of the present invention the dimensions of the length of the anterior wall 518 of the wedge 406 is 0.2 mm, however it is understood that the length may vary in other embodiments up to 0.5 mm.

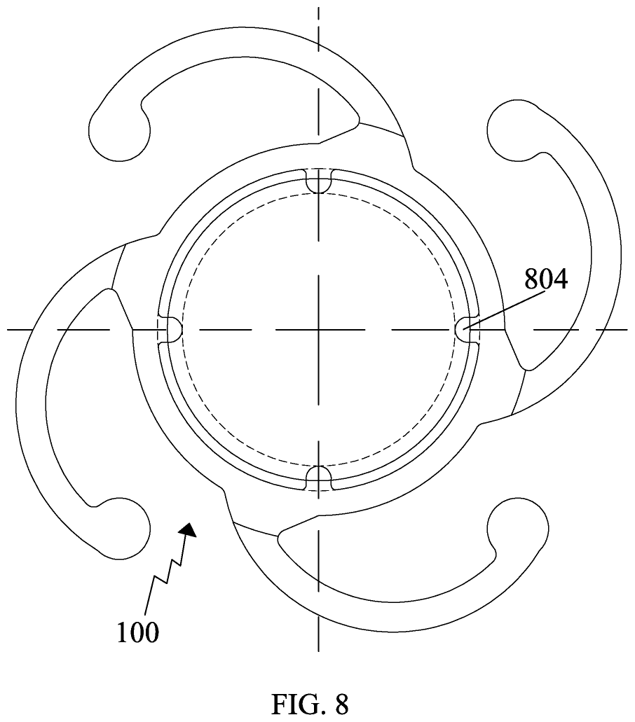

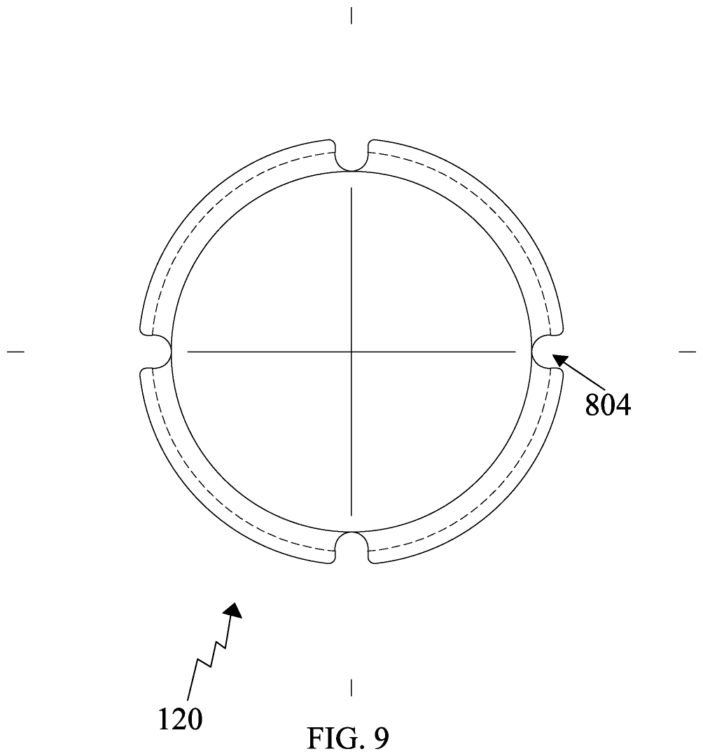

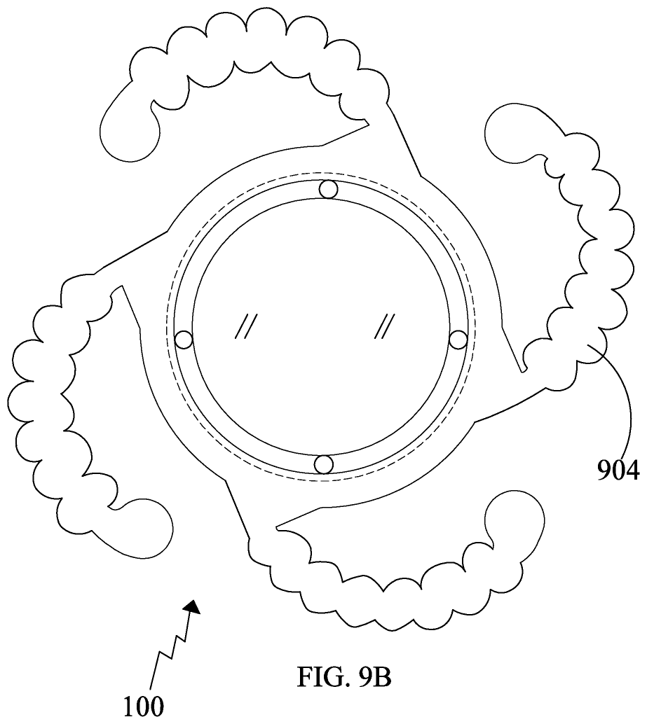

Referring to FIGS. 8 and 9, one more embodiment of the present invention is described. The optic includes a plurality of pairs of opposed notches 804. The opposed notches 804 are defined along the peripheral edge of the optic 104.

In another embodiment of the optic haptic assembly shown in FIGS. 8, the first end of the arms 108 are rounded tip less and the second end of the arms 108 include base 110. In this one embodiment the arms 108 are preferably made of Polymethyl methacrylate (PMMA) material. It is however noted that in this embodiment the design and material of base 110, haptic ring 106 and optic 104 remains the same.

Referring to FIG. 9B, another embodiment of the intraocular lens assembly 100 has a plurality of rounded protrusions 904 along both edges of each of the arms 108. However, it is understood that in this embodiment the haptic 104 is in accordance with the preferred embodiment of the present invention.

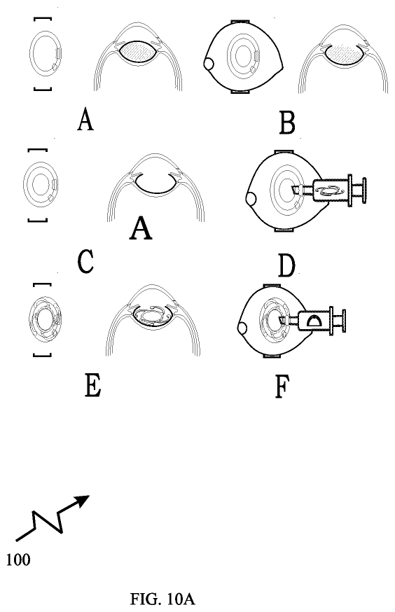

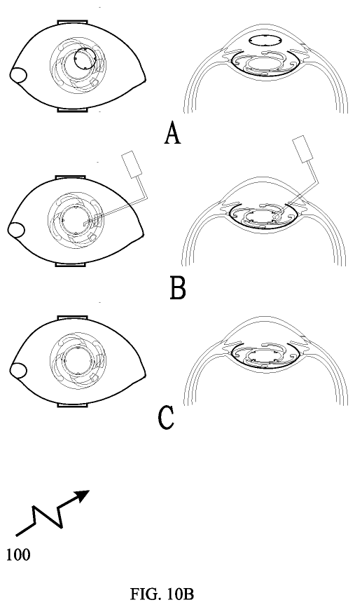

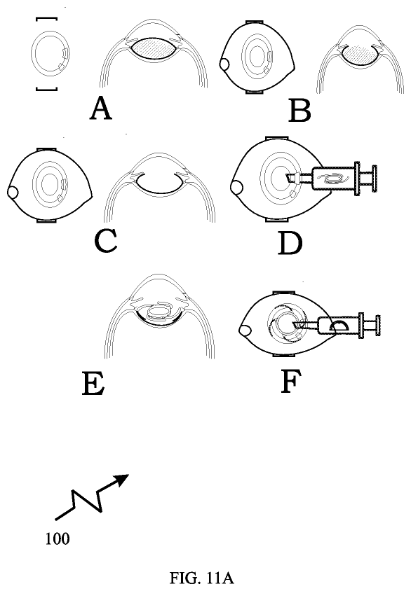

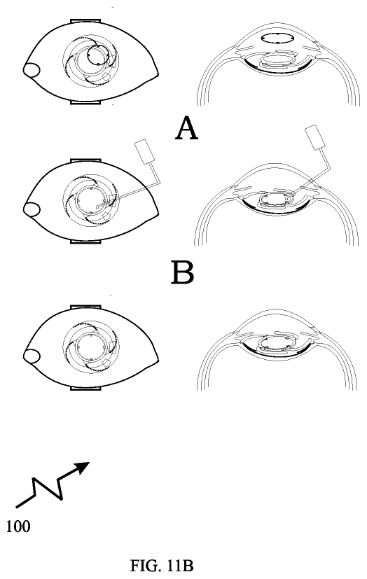

Now referring to FIGS. 10A, 10B and 11A, 11B a preferred method of positioning the optic haptic assembly 100 of the present invention in the human eye during cataract surgery is described. In a first step, cataract is removed and the capsular bag is cleaned, further viscoelastic substance is inserted in the anterior chamber and the capsular bag to open it completely. In next step, as shown in FIG. 10A, the haptic 102 is injected inside the eye with injector and cartridge. In step ‘E’ as shown in FIG. 10A, after the haptic 102 unfolds the haptic arms 108 are manipulated to be placed inside the capsular bag. This leads to placement of the haptic 102 in the capsular bag and the haptic arms 108 placed at the inside periphery or angle of the capsular bag at four points which are along perpendicular meridians.

In step ‘F’ as shown in FIG. 10A; the optic 104 is inserted inside the eye with injector and cartridge. In step ‘A’ as shown in FIG. 10B, after the optic104 unfolds the optic 104 is brought in front of the opening in the haptic 102 for the optic using a Sinsky hook. In next step, the lower surface of the optic 104 and the groove on optic 404 helps in aligning the optic on the haptic. In next step, after fine alignment of the optic 104 it is pressed downwards at the center using an iris repositor, so that the optic 104 snap fits in the haptic 102. In last step, each quadrant between the holes 112 or notches 804 of optic is again pressed downwards to confirm 360° placement of the optic periphery below the wedge 406.

Now a preferred method of changing an existing optic by a new optic in an existing optic haptic assembly previously inserted in a human eye is described. Saline is injected through the notches 804 or holes 112 in the optic. This leads to separation of the posterior capsule from the optic 104. With the sinsky hook placed in the notches 804 or holes 112 the optic is pulled upwards to release it from the adjacent wedge 406 of the haptic 102. This is repeated for all the notches or holes of optic releasing it from the haptic. The optic 104 is now lifted above the haptic 102. The optic is cut in to multiple pieces and removed from the eye one by one. The new optic 104 to be placed inside the eye is injected using injector and cartridge. After the optic unfolds the optic is brought in front of the opening in the haptic for the optic using a Sinsky hook.

The lower surface of the optic and the groove on optic 404 helps in aligning the optic on the haptic. After fine alignment of the optic, the optic is pressed downwards at the center using a iris repositor or forceps, so that the optic snap fits in the haptic. Each quadrant between the holes 112 or notches 804 of optic is again pressed downwards to confirm 360° placement of the optic periphery below the wedge 406.

Referring to second embodiment of the present invention steps for positioning the optic haptic assembly 100 of the present invention in the human eye during cataract surgery is described in cases where capsular bag integrity is compromised as in case of posterior capsular rupture. In a first step ‘D’ as shown in FIG. 11A, the haptic 102 is injected inside the eye with injector and cartridge. In next step, after the haptic 102 unfolds the haptic arms 108 are manipulated to be placed in the ciliary sulcus. This leads to placement of the haptic 102 in the ciliary sulcus above the capsular bag and the haptic arms 108 are placed between the iris base and ciliary body.

In step ‘F’ as shown in FIG. 11A, the optic 104 is inserted inside the eye with injector and cartridge. In step ‘A’ as shown in FIG. 11B, after the optic 104 unfolds the optic 104 is brought in front of the opening in the haptic 102 for the optic 104 using a Sinsky hook. The lower surface of the optic 104 and the groove 404 on optic helps in aligning the optic 104 on the haptic 102. After fine alignment of the optic 104 it is pressed downwards at the center using forceps, so that the optic 104 snap fits in the haptic 102. Each quadrant between the holes 112 or notches 804 of optic 104 is again pressed downwards to confirm 360° placement of the optic periphery below the wedge 406.

In accordance with an embodiment of the present invention, the locking arrangement provides an anteroposterior and rotational stability to the optic 104 and further prevents optic from displacing due to vitreous pressure from the posterior side. In the embodiment of the present invention, the number of dialing holes 112 or notches 804 of the optic 104 may vary i.e. may be from one to four in number. But inaccordance with this preferred embodiment, there are four optic holes 112 or four notches 804 for better control and manipulation.

In accordance with the embodiment of the present invention, the haptic 102 with four C looped haptic arms 108 gives a four point fixation along perpendicular meridians which gives better centration of the intraocular lens assembly 100. Hence the four point fixation is preferred in this embodiment.

The locking arrangement of the IOL assembly 100 of the present invention advantageously achieves a good anteroposterior stability due to the wedge 406 on haptic 102 and rotational stability of the changeable optics due to the step 408 on haptic 102 and corresponding step 600 on optic 104. In the present IOL assembly no significant alteration in the surgical procedure will be required apart from injecting the intraocular lens in 2 parts i.e. 1st injecting the haptic, aligning it inside the eye and then injecting the optic and then aligning and locking it. The size and shape of the anterior capsulorhexis has minimal effect on centration and stability of the intraocular lens due to its design leading to better optical results. The haptic arms are angulated from the haptic plane in the range of 0-10 degrees which decreases posterior capsular opacification rate by tightly opposing the posterior capsule with the optic and also prevents rise in intraocular pressure by keeping a gap between iris and the optic. 14. The rounded protrusion 904 along both edges of the arms 108, provide better fixation of the haptic 102.

The embodiments were chosen and described in order to best explain the principles of the present invention and its practical application, to thereby enable others, skilled in the art to best utilize the present invention and various embodiments with various modifications as are suited to the particular use contemplated.

It is understood that various omission and substitutions of equivalents are contemplated as circumstance may suggest or render expedient, but such are intended to cover the application or implementation without departing from the spirit or scope of the present invention.

1. An intraocular lens assembly (100) comprising:
a haptic (102) having a ring (106), a plurality of arcuate arms (108), and a plurality of haptic arm bases (110), each of the arms (108) being connected with the ring (106) by a respective base (110);
an optic (104) having at least two pairs of opposed holes (112), the holes (112) being positioned in the close proximity with an outer peripheral edge of the optic (104);
the optic (104) being removably positionable in the haptic (102) by a snap fit lock forming the intraocular lens assembly; and
at least two positions of the intraocular lens assembly (100), a first unlocked position wherein haptic (102) is dissembled from the optic (104), and a second locked position wherein the optic (104) is snap fitted in the haptic (102).

2. The intraocular lens assembly (100) as claimed in claim 1, wherein each of the arcuate arms (108) has a first end that includes a rounded tip (114), and the respective second end being connected to the respective base (110).

3. The intraocular lens assembly (100) as claimed in claim 1, wherein the haptic (102) includes a first groove (402), the first groove (402) being configured to run along the inner circumference of the haptic (102).

4. The intraocular lens assembly (100) as claimed in claim 3, wherein the first groove (402) includes a wedge (406) and a step (408).

5. The intraocular lens assembly (100) as claimed in claim 4, wherein the wedge (406) includes a posterior wall (516) and an anterior wall 518 that meet at apex on inner circumference of haptic (102).

6. The intraocular lens assembly 100 as claimed in claim 4, wherein the step (408) has an anterior vertical first wall (510), a horizontal second wall (512) and a posterior vertical wall (514).

7. The intraocular lens assembly (100) as claimed in claim 1, wherein the haptic (102) of assembly (100) has anterior first wall (510) and the horizontal second wall (512) that are perpendicular to each other.

8. The intraocular lens assembly (100) as claimed in claim 1, wherein the, horizontal second wall (512) and posterior third wall (514) are perpendicular to each other.

9. The intraocular lens assembly (100) as claimed in claim 1, wherein the optic (104) includes a second groove (404) that has a step (600), and a hatch (602) that includes a planer base (604), a peripheral wall (606) and a top wall (608).

10. The intraocular lens assembly (100) as claimed in claim 9, the step (600) is defined by a peripheral wall (606), the posterior and inner vertical third wall (610), and a horizontal second wall (604).

11. The intraocular lens assembly (100) as claimed in claim 1, wherein the second groove (404) is slideably positionable inside the first groove (402) defining the lock (400).

12. The intraocular lens assembly (100) as claimed in claim 1, wherein the base (604) of optic (104) rests on second horizontal wall (512) of haptic (102) in the second locked position of the optic haptic assembly.

13. The intraocular lens assembly as claimed in claim 1, wherein the peripheral wall (606) of the optic (104) opposes to first wall (510) of the haptic (102).

14. The intraocular lens assembly as claimed in claim 1, wherein the vertical wall (610) of optic (104) opposes to third wall (514) of haptic (102).

15. The intraocular lens assembly as claimed in claim 1, wherein the top (608) of the hatch (602) touches the posterior wall (516) of the wedge (406).

16. The intraocular lens assembly as claimed in claim 1, wherein the optic (104) has at least two opposed pair of notches (804) instead of holes (112), the notches (804) being positioned in the close proximity with an outer peripheral edge of the optic (104).

17. The intraocular lens assembly as claimed in claim 1, wherein the haptic (102) has a plurality of rounded protrusions (904) along both edges of the arms 108), the rounded protrusions provide better fixation of the haptic (102).
